(19) United States
(12) Patent Application Publication
(10) Pub. No.: US 2013/0190822 A1
(43) Pub. Date: Jul. 25, 2013

(54) SOINAL IMPLANT SYSTEM AND METHOD

(71) Applicant: Alan Rezach, Atoka, TN (US)

(72) Inventor: Alan Rezach, Atoka, TN (US)

(73) Assignee: Warsaw Orthopedic, Inc., Warsaw, IN (US)

(21) Appl. No.: 13/354,575

(22) Filed: Jan. 20, 2012

(51) Int. Cl. A61B 17/70; A61B 17/00; A61B 17/56

(52) U.S. Cl. 606/264; 606/86 A; 606/1

(57) ABSTRACT

An implant reducer comprises a first arm including a first member and a second member. The second member includes an inner surface that defines a first implant engaging portion. A second arm includes a first member and a second member. The second member of the second arm includes a connector and a second implant engaging portion. The first members are connected and the connector is movably disposed in a longitudinal cavity. The first members are relatively rotatable to move the arms between a first configuration, a second configuration such that the first implant engaging portion is disposed adjacent the second implant engaging portion and a third configuration such that the connector translates relative to the inner surface and the second implant engaging portion translates relative to the first implant engaging portion. Methods of use are disclosed.

### TECHNICAL FIELD

The present disclosure generally relates to medical devices for the treatment of spinal disorders, and more particularly to a spinal implant system and method, which has the capability to provide stability while reducing stress on spinal elements.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders, Surgical treatment of these spinal disorders includes discectomy, laminectomy, fusion and implantable prosthetics. During surgical treatment, one or more rods may be fastened to the exterior of two or more vertebral members to provide stability to a treated region. This disclosure describes an improvement over these prior art technologies.

### SUMMARY

Accordingly, a spinal implant system is provided. In one embodiment, in accordance with the principles of the present disclosure, an implant reducer comprises a first arm including a first member and a second member extending from the first member. The second member includes an inner surface that defines a longitudinal cavity and a first implant engaging portion. A second arm includes a first member and a second member extending from the first member of the second arm. The second member of the second arm includes a connector and a second implant engaging portion. The first members are connected and the connector is movably disposed in the longitudinal cavity. The first members are relatively rotatable to move the arms between at least a first configuration such that the first implant engaging portion and the second implant engaging portion are spaced apart, a second configuration such that the first implant engaging portion is disposed adjacent the second implant engaging portion and a third configuration such that the connector translates relative to the inner surface and the second implant engaging portion translates relative to the first implant engaging portion.

In one embodiment, in accordance with the principles of the present disclosure, a spinal implant system comprises an implant reducer including a first arm having a proximal member and a distal member. The distal member includes an inner surface that defines a longitudinal cavity and a first implant engaging portion. A second arm includes a proximal member and a distal member. The distal member of the second arm includes a connector and a second implant engaging portion. The proximal members are connected and the connector is movably disposed in the longitudinal cavity. A first implant includes a proximal portion defining an implant cavity and a distal portion configured for penetrating tissue. The first implant engaging portion is configured to engage the proximal portion of the first implant. A second implant includes a vertebral rod configured for disposal with the implant cavity. The second implant engaging portion is configured to engage the second implant. The first members are relatively rotatable to move the arms between at least a first configuration such that the first implant engaging portion and the second implant engaging portion are spaced apart, a second configuration such that the first implant engaging portion engages the proximal portion of the first implant and is disposed adjacent the second implant engaging portion and a third configuration such that the connector translates relative to the inner surface and the second implant engaging portion translates relative to the first implant engaging portion.

In one embodiment, the spinal implant system comprises a rod reducer comprising a first arm including a proximal member connected to a distal member via a first pivot such that the distal member is rotatable relative to the proximal member. The distal member includes an inner surface that defines a longitudinal slot and a pair of spaced apart extensions including inwardly disposed pins. A second arm includes a proximal member connected to a distal member via a second pivot such that the distal member of the second arm is rotatable relative to the proximal member of the second arm. The distal member of the second arm includes a connector and a pair of spaced apart extensions including concave surfaces. The proximal members are connected and the connector is movably disposed in the longitudinal slot. A bone fastener includes a receiver defining an implant cavity and a threaded distal shaft. The receiver further includes an outer surface that defines an axial cavity including a transverse opening configured for passage of the pins. A vertebral rod is configured for disposal with the implant cavity. The concave surfaces are configured to engage an outer surface of the vertebral rod. The proximal members are relatively rotatable to move the arms between a first configuration such that the extensions of the arms are spaced apart, a second configuration such that the pins are disposed in the axial cavity and the concave surfaces engage the outer surface of the vertebral rod, and the extensions of the first arm are disposed adjacent the extensions of the second arm, and a third configuration such that the connector translates relative to the inner surface and the extensions of the first arm translate relative to the extensions of the second arm such that the implant cavity rotates relative to the vertebral rod.

### BRIEF DESCRIPTION OF THE DRAWINGS

The present disclosure will become more readily apparent from the specific description accompanied by the following drawings, in which:

FIG. 1 is a perspective view of one embodiment of a component of a spinal implant system in accordance with the principles of the present disclosure;

FIG. 2 is a perspective view of one embodiment of a component of the spinal implant system in accordance with the principles of the present disclosure;

FIG. 3 is a perspective view of one embodiment of a spinal implant system in accordance with the principles of the present disclosure;

FIG. 4 is a perspective view of the spinal implant system shown in FIG. 3; and

FIG. 5 is a perspective view of the spinal implant system shown in FIG. 3.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine. It is envisioned that the surgical system can include an instrument system having extenders, reducers and translators, which can be used to introduce a vertebral construct such as a rod to a bone fastener such as a bone anchor or bone screw. For example, a rod reducer can include bone anchor attachment features on one or both sides of the instrument. It is contemplated that the system may be used with a reducer assembly to introduce a rod into a bone fastener.

In one embodiment, the spinal implant system includes a rod reducer that includes a break-away pivot point to allow rod reduction into an implant, such as a head of a bone fastener. It is contemplated that the break away pivot point may include a biasing member, which may be spring loaded. This configuration provides maneuverability for reducing a rod with a bone fastener. It is contemplated that the spinal implant system can be employed for reducing a rod that is medial/lateral to an implant such as a bone fastener.

In one embodiment, the rod reducer is configured to engage a pivot point disposed higher and/or more proximal on the head of a hone fastener to allow easier docking of the component parts. In one embodiment, the head of the bone fastener includes an elongated slot or emboss with an opening so that the rod reducer can pivotally attach to the head of the bone fastener.

In one embodiment, the rod reducer is disposable in one or a plurality of configurations for engagement with one or a plurality of implants. For example, in a first configuration, the rod reducer can be disposed in an open position. For example, in a second configuration, a handle of the rod reducer is manipulated or squeezed to align the implant(s) and/or pull a rod towards the head of a bone fastener. For example, in a third configuration, a handle is manipulated or squeezed to translate the rod reducer and seat the rod with the head of the bone fastener. It is envisioned that a set screw can be loaded into the head of the bone fastener and tightened against the rod.

It is contemplated that one or all of the components of the spinal implant system may be disposable, peel-pack, pre-packed sterile devices. One or all of the components of the system may be reusable. The system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

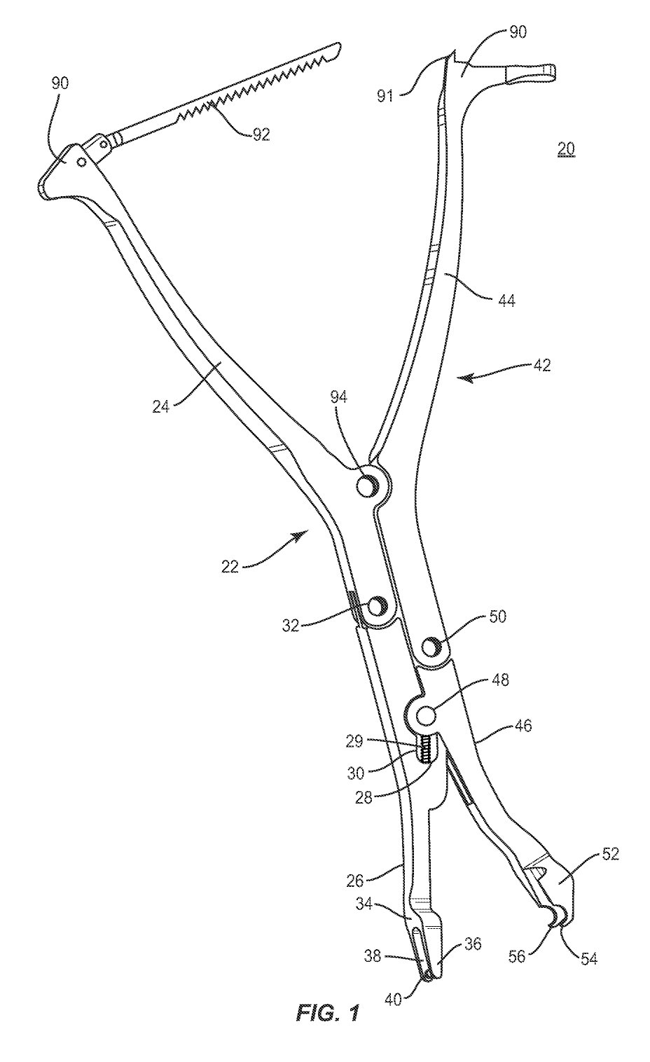

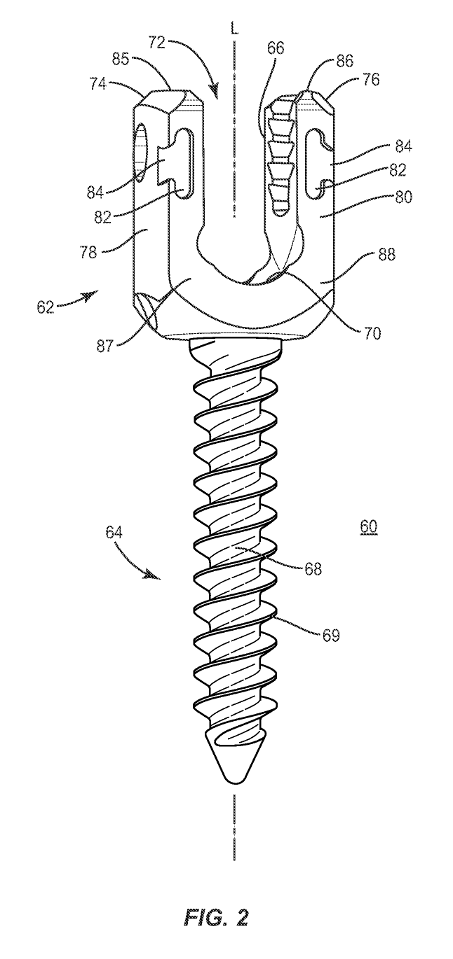

The following discussion includes a description of a surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a system, such as, for example, a spinal implant system 10 in accordance with the principles of the present disclosure.

The components of spinal implant system 10 can be fabricated from biologically acceptable materials suitable for medical applications, including metals, synthetic polymers, ceramics, bone material, tissue and/or their composites, depending on the particular application and/or preference of a medical practitioner. For example, the components of spinal implant system 10, individually or collectively, can be lubricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), lubric, silicone, polyurethane, silicone-polyurethane copolymers, polymeric rubbers, polyolefin rubbers; hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

Spinal implant system 10 is employed, for example, with an open or mini-open, minimal access and/or minimally invasive including percutaneous surgical technique to deliver and introduce an implant, such as, for example, a vertebral construct such as a spinal rod to an implant, such as, for example, a bone fastener, at a surgical site within a body of a patient, for example, a section of a spine. In one embodiment, the components of spinal implant system 10 are configured to position the vertebral rod into engagement with the bone fastener for a surgical treatment to treat various spine pathologies, such as those described herein.

Spinal implant system 10 includes an implant reducer, such as, for example, a rod reducer 20. Rod reducer 20 includes a first arm 22 and a second arm 42. It is contemplated that the cross section and/or overall configuration of arm 22 and/or arm 42 may be variously configured, such as, for example, round, oval, oblong, square, rectangular, polygonal, irregular, uniform, non-uniform, offset, staggered, tapered, consistent or variable, depending on the requirements of a particular application.

First arm 22 includes a first member, such as, for example, a proximal member 24 and a second member, such as, for example, a distal member 26. Proximal member 24 and distal member 26 are connected by a first pivot 32 such that distal member 26 is rotatable relative to proximal member 24. Distal member 26 includes an inner surface 28 that defines a cavity, such as, for example, a longitudinal slot 30. Longitudinal slot 30 extends axially and at an acute angle relative to a longitudinal axis of arm 22 and is configured for disposal of a connector, discussed below. It is contemplated that slot 30 may be disposed in alternate orientations relative to the longitudinal axis of arm 22, such as, for example, parallel, transverse and/or other angular orientations such as acute or obtuse, co-axial and/or may be offset or staggered. It is envisioned that first arm 22 may include one or a plurality of proximal and/or distal members that may be connected for relative pivoting and/or non-pivoting movement.

Distal member 26 includes a first implant engaging portion, such as, for example, a pair of spaced apart extensions 36, 38. Each of extensions 36, 38 include at least one projection, such as, for example, opposing pins 40. Each pin 40 has an inwardly disposed configuration oriented to engage an implant, discussed below. It is contemplated that each extension may include one or a plurality of projections. It is further contemplated that one or each of the projections may be disposed in alternate orientations relative to extensions 36, 38, such as, for example, parallel, transverse and/or other angular orientations such as acute or obtuse, co-axial and/or may be offset or staggered. It is envisioned that the extensions may include a gripping surface, which may include a projection and/or other surface configurations to enhance engagement with an implant, such as, for example, rough, arcuate, undulating, mesh, porous, semi-porous, dimpled and/or textured according to the requirements of a particular application.

Second arm 42 includes a first member, such as, for example, a proximal member 44 and a second member, such as, for example, a distal member 46. Proximal member 44 and distal member 46 are connected by a second pivot 50 such that distal member 46 is rotatable relative to proximal member 44. It is envisioned that second arm 42 may include one or a plurality of proximal and/or distal members that may be connected for relative pivoting and/or non-pivoting movement.

Distal member 46 includes a connector 48 being movably disposed in longitudinal slot 30. At least a portion of connector 48 has a cylindrical configuration oriented transverse to a longitudinal axis of second arm 42. Connector 48 slidably engages inner surface 28 and has a movable stop limit at a distal end of slot 30. A biasing member, such as, for example, a spring 29 is disposed with slot 30 and in a configuration to engage connector 48. Spring 29 provides a resilient bias to connector 48 in a proximal direction and/or toward a handle 90. It is contemplated that the biasing member may include an elastomeric member, clip, leaf spring, gravity induced configuration, pneumatic configuration, hydraulic configuration and/or manual lever. It is further contemplated that connector 48 may be non-biased within slot 30.

Distal member 46 includes a second implant engaging portion, such as, for example, a pair of spaced apart extensions 54, 56. Each of extensions 54, 56 include a concave surface that engage an implant. Each concave surface is oriented to engage and support an implant, discussed below. It is contemplated that distal member 46 may include one or a plurality of extensions. It is further contemplated that one or each of the concave surfaces may be disposed in alternate orientations relative to extensions 54, 56, such as, for example, parallel, transverse and/or other angular orientations such as acute or obtuse, co-axial and/or may be offset or staggered. It is envisioned that the concave surfaces may include a gripping surface, which may include configurations to enhance engagement with an implant, such as, for example, rough, arcuate, undulating, mesh, porous, semi-porous, dimpled and/or textured according to the requirements of a particular application.

Proximal members 24 and 44 are connected to portions of handle 90 for operation of rod reducer 20. Handle 90 is elongated and includes a pawl 91 for engaging a toothed bar 92. Proximal members 24 and 44 are connected via a pivot 94 configured to facilitate rotation of proximal member 44 of second arm 42 relative to proximal member 24 of first arm 22.

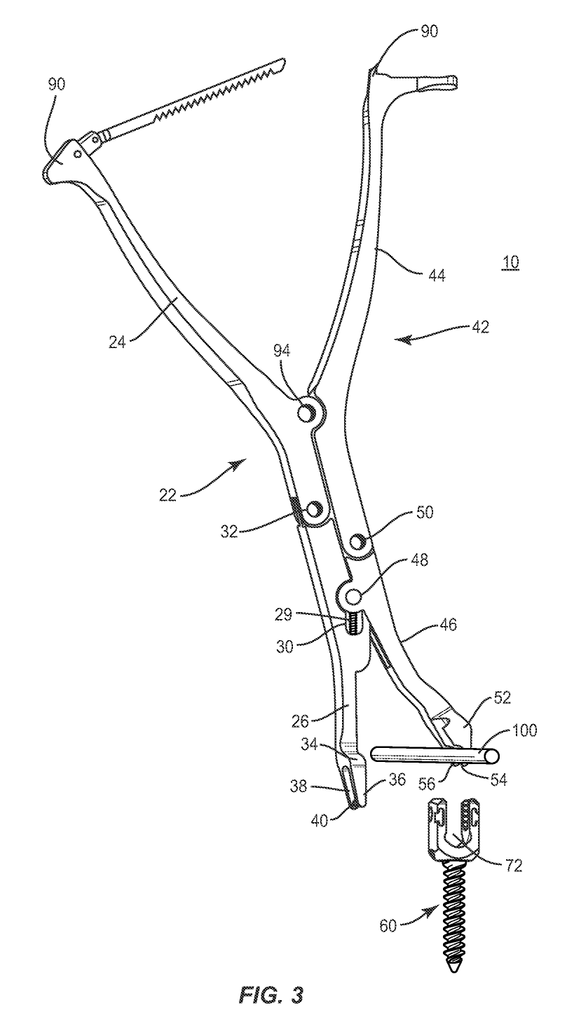

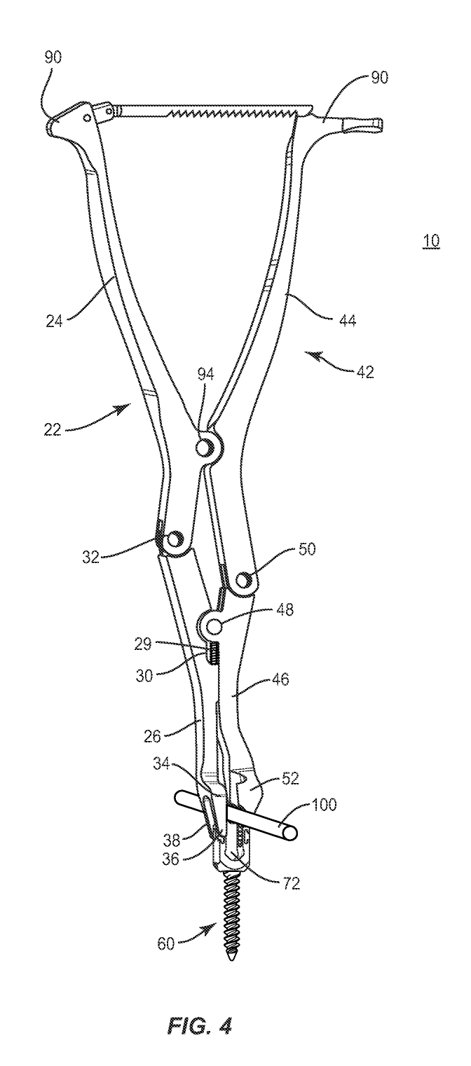

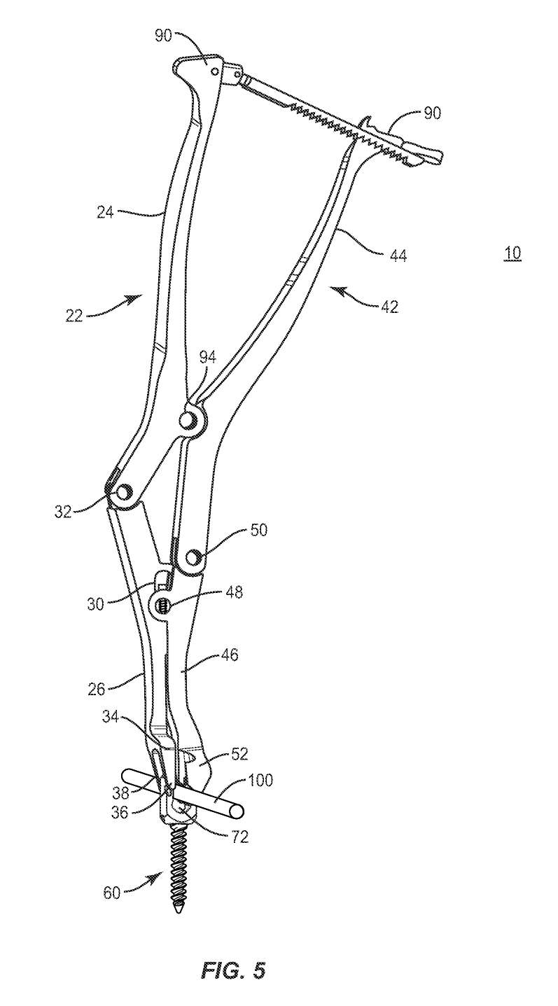

In operation, as shown in FIGS. 3-5, rod reducer 20 is movable between a first configuration (FIG. 3), a second configuration (FIG. 4) and a third configuration (FIG. 5). It is contemplated that rod reducer 20 is disposable in one or a plurality of configurations.

In the first configuration, such as, for example, an open position, arms 22, 42 and distal members 26, 46 are spaced apart. Pivot 32 and pivot 50 are disposed in close proximity such that proximal member 24 is disposed in longitudinal alignment with distal member 26, and proximal member 44 is disposed in longitudinal alignment with distal member 46. Extensions 36, 38 and extensions 54, 56 of arms 22, 42, respectively, are spaced apart.

Handle 90 is manipulated by a practitioner such that proximal members 24, 44 are squeezed together in relative rotation and/or pivoting relation to move arms 22, 42 to initiate manipulation of implants from the first configuration. As handle 90 is further manipulated and squeezed together from the first configuration, pivot 94 pivots proximal members 24, 44 such that pivots 32, 50 space apart. Distal member 26 is caused to rotate relative to proximal member 24 and distal member 46 is caused to rotate relative to proximal member 44. Connector 48 is disposed at a proximal most position in slot 30. Pins 40 are caused to engage a first implant, as described below, and the concave surfaces of extensions 54, 56 engage an outer surface of a second implant such that rod reducer 20 is disposed in the second configuration, such as, for example, an intermediate position. Extensions 36, 38 of first arm 22 are disposed adjacent extensions 54, 56 of second arm 42.

As handle 90 is further manipulated and squeezed together from the second configuration, pivots 32, 50 cause further relative rotation of members 24, 26 and members 44, 46. Connector 48 translates axially within slot 30 relative to inner surface 28. Distal member 46 translates axially relative to distal member 26 and extensions 36, 38 translate axially relative to extensions 54, 56 such that rod reducer 20 is disposed in the third configuration, such as, for example, a closed position. Toothed bar 92 may be engaged with pawl 91 to lock rod reducer 20 in a position between and including the first and third configurations. The implants are caused to rotate for seating thereof as will be described. Spring 29 engages connector 48 such that connector 48 is resiliently biased from the third configuration to the second configuration and/or the first configuration, upon release of handle 90 to release the implants from engagement with rod reducer 20.

A first implant, such as, for example, a bone fastener 60 comprises a proximal portion, such as, for example, a receiver 62 and a distal portion, such as, for example, an elongated shaft 64 configured for penetrating tissue. It is envisioned that spinal implant system 10 may include one or a plurality of fixation elements.

Shaft 64 has a cylindrical cross section configuration and includes an outer surface 68 having an external threaded form 69. It is contemplated that thread form 69 may include a single thread turn or a plurality of discrete threads. It is further contemplated that other engaging structures may be located on shaft 64, such as, for example, a nail configuration, barbs, expanding elements, raised elements and/or spikes to facilitate engagement of shaft 64 with tissue, such as, for example, vertebrae.

It is envisioned that all or only a portion of shaft 64 may have alternate cross section configurations, such as, for example, oval, oblong, triangular, square, polygonal, irregular, uniform, non-uniform, offset, staggered, undulating, arcuate, variable and/or tapered. It is contemplated that outer surface 68 may include one or a plurality of openings. It is contemplated that all or only a portion of outer surface 68 may have alternate surface configurations to enhance fixation with tissue such as, for example, rough, arcuate, undulating, mesh, porous, semi-porous, dimpled and/or textured according to the requirements of a particular application. It is envisioned that all or only a portion of shaft 64 may be disposed at alternate orientations, relative to a longitudinal axis L of bone fastener 60, such as, for example, transverse, perpendicular and/or other angular orientations such as acute or obtuse, co-axial and/or may be offset or staggered. It is further envisioned that all or only a portion of shaft 64 may be cannulated.

It is contemplated that shaft 64 may be made for attachment to bone, such as cervical, thoracic, lumbar and or sacral vertebral bone structures, or other tissues. In one embodiment, shaft 64 may be a screw, or could also be alternatively configured, for example, as a vertebral hook or clamp. It is envisioned that threads 69 may be a cancellous thread, of a configuration suited for implantation into a vertebra. It is further contemplated that threads 69 may be self-tapping or intermittent, or may have more than one crest winding about shaft 68. In one embodiment, outer surface 68 may include an opening for accommodating a tool (not shown) for gripping or turning bone fastener 60.

Receiver 62 defines an inner surface that includes a wall surface 66 and a base surface 70. The inner surface of receiver 62 defines a cavity, such as, for example, a U-shaped passageway 72 configured for disposal of an implant. Receiver 62 has a first arm 74 and a second arm 76 extending proximally from shaft 64 along axis L. Arms 74, 76 include wall surface 66 and define at least a portion of U-shaped passageway 72. Arm 74 includes an outer surface 78 and arm 76 includes an outer surface 80.

It is envisioned that all or only a portion of passageway 72 may have alternate cross section configurations, such as, for example, oval, oblong, triangular, square, polygonal, irregular, uniform, non-uniform, offset, staggered and/or tapered. It is contemplated that arm 72 and/or arm 74 may include one or a plurality of openings. It is envisioned that arm 72 and/or arm 74 may be disposed at alternate orientations, relative to axis L, such as, for example, transverse, perpendicular and/or other angular orientations such as acute or obtuse, co-axial and/or may be offset or staggered.

Wall surface 66 includes a thread form located adjacent first arm 72 and a thread form located adjacent second arm 74. The thread forms are configured for engagement with a coupling member, such as, for example, a setscrew to attach, fix and/or lock components, such as a spinal rod, of spinal implant system 10 with bone fastener 60.

Outer surfaces 78, 80 each define attaching cavities, such as, for example, axial slots 82 configured to receive a portion of rod reducer 20 for attachment thereto and seating of a spinal rod with bone fastener 60. Arms 72, 74 each include a pair of axial slots 82 disposed on lateral sides thereof for disposal of pins 40. Each axial slot 82 includes a transverse opening 84 configured for passage of pins 40. Axial slots 82 are disposed on arms 72, 74 adjacent proximal most ends 85, 86 and extend axially towards distal most ends 87, 88 to an intermediated position on outer surfaces 78, 80. Transverse openings 84 are disposed such that pins 40 can slide therethrough into axial slot 82 and the walls of slots 82 retain pins 40 therein and bone fastener 60 can be manipulated for seating a spinal rod therewith. This configuration facilitates seating a spinal rod in passageway 72 and such engagement of pins 40 with slots 82 allows rod reducer 20 to pivot or rotate receiver 62, thereby enabling reduction of the spinal rod.

it is envisioned that outer surfaces 78, 80 may include alternate fixation configurations with rod reducer 20, such as, for example, friction fit, pressure fit, locking protrusion/recess, locking keyway and/or adhesive. It is contemplated that all or only a portion of outer surfaces 78, 80 may have alternate surface configurations to enhance fixation with rod reducer 20, such as, for example, rough, arcuate, undulating, mesh, porous, semi-porous, dimpled and/or textured according to the requirements of a particular application.

A second implant, such as for example, a vertebral rod 100 is provided. Vertebral rod 100 is configured for disposal in passageway 72. The concave surfaces of extensions 54 and 56 are configured to engage an outer surface of vertebral rod 100. Vertebral rod 100 is substantially cylindrical and generally configured to fit within receiver 62. Vertebral rod 100 may also have portions of its surface roughened or otherwise modified to introduce friction between the outer surface of vertebral rod 100 and the inner surface of receiver 62, thereby further stabilizing the connection between bone fastener 60 and vertebral rod 100.

In assembly and use, spinal implant system 10 is employed with a surgical procedure for treatment of a spinal disorder affecting a section of a spine of a patient, as discussed herein. In use, to treat the affected section of the spine, a medical practitioner obtains access to a surgical site including vertebrae (not shown) in any appropriate manner, such as through incision and retraction of tissues. It is envisioned that spinal implant system 10 may be used in any existing surgical method or technique including open surgery, mini-open surgery, minimally invasive surgery and percutaneous surgical implantation, whereby the vertebrae is accessed through a micro-incision, or sleeve that provides a protected passageway to the area. Once access to the surgical site is obtained, the particular surgical procedure is performed for treating the spinal disorder. Spinal implant system 10 is then employed to augment the surgical treatment.

One or more bone fasteners 60 are delivered to a surgical site fir fixation with vertebrae and a vertebral rod 100 is delivered to the surgical site adjacent thereto. A medical practitioner positions vertebral rod 100 adjacent to bone fasteners 60 for seating rod 100 with bone fastener 60. Rod reducer 20 is introduced to reduce or force vertebral rod 100 into passageway 72 of bone fastener 60. With arms 22 and 42 in an unstressed state, i.e. biased away from each other or otherwise spread apart, distal ends 26 and 46 are also spread apart. As shown in FIG. 3, rod reducer 20 is in the first configuration such that arms 22, 42 and distal members 26, 46 are spaced apart. Extensions 36, 38 and extensions 54, 56 of arms 22, 42, respectively, are spaced apart.

Handle 90 is manipulated by the medical practitioner and squeezed together from the first configuration. As shown in FIG. 4, pins 40 pass through transverse openings 84 and are disposed within slots 82 to engage bone fastener 60 such that the surfaces of slots 82 retain pins 40 therein. The concave surfaces of extensions 54, 56 engage an outer surface of vertebral rod 100 such that rod reducer 20 is disposed in the second configuration.

As handle 90 is further manipulated and squeezed together from the second configuration, connector 48 translates axially within slot 30 relative to inner surface 28. Distal member 46 translates axially relative to distal member 26 and extensions 36, 38 translate axially relative to extensions 54, 56 such that rod reducer 20 is disposed in the third configuration, as shown in FIG. 5. Pins 40 engage the surfaces of slots 82 to pivot or rotate receiver 62 and the concave surfaces of extensions 54, 56 drive vertebral rod 100 into passageway 72. This configuration facilitates seating vertebral rod 100 in passageway 72 and such engagement of pins 40 with slots 82 allows rod reducer 20 to pivot or rotate receiver 62, thereby enabling reduction of vertebral rod 100. Spring 29 engages connector 48 such that connector 48 is resiliently biased from the third configuration to the second configuration and/or the first configuration, upon release of handle 90 to release bone fastener 60 and vertebral rod vertebral rod 100 from engagement with rod reducer 20.

It is envisioned that pivoting of rod reducer 20 and/or squeezing of handle 90 may be continued until vertebral rod 100 is seated within receiver 62. Such pivoting may force vertebral rod 100 generally Obliquely with respect to axis L, or in a direction that has at least a component parallel to axis L.

Once vertebral rod 100 is positioned in bone fastener 60 according to the requirements of a procedure, vertebral rod 100 can be locked into bone fastener 60 using a set screw provided with bone fastener 60. The medical practitioner may then seat or reduce vertebral rod 100 with respect to other bone fasteners 60, according to the requirements of a particular procedure.

Spinal implant system 10 can be used with various bone screws, mono-axial screws, pedicle screws or multi-axial screws used in spinal surgery. In one embodiment, spinal implant system 10 includes an agent, which may be disposed, packed or layered within, on or about the surfaces of bone fastener 60. It is envisioned that the agent may include bone growth promoting material, such as, for example, bone graft to enhance fixation of the fixation elements with vertebrae V.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of spinal implant system 10 can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

it is envisioned that the use of microsurgical and image guided technologies may be employed to access, view and repair spinal deterioration or damage, with the aid of spinal implant system 10.

it will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An implant reducer comprising:
a first arm including a first member and a second member extending from the first member, the second member including an inner surface that defines a longitudinal cavity and a first implant engaging portion; and
a second arm including a first member and a second member extending from the first member of the second arm, the second member of the second arm including a connector and a second implant engaging portion, the first members being connected and the connector being movably disposed in the longitudinal cavity,
wherein the first members are relatively rotatable to move the arms between at least a first configuration such that the first implant engaging portion and the second implant engaging portion are spaced apart, a second configuration such that the first implant engaging portion is disposed adjacent the second implant engaging portion and a third configuration such that the connector translates relative to the inner surface and the second implant engaging portion translates relative to the first implant engaging portion.

2. An implant reducer as recited in claim 1, further comprising a handle including the first members.

3. An implant reducer as recited in claim 1, wherein the first members are connected via a pivot configured to facilitate rotation of the first member of the second arm relative to the first member of the first arm.

4. An implant reducer as recited in claim 1, wherein the first arm includes a pivot configured to facilitate rotation of the second member of the first arm relative to the first member of the first arm.

5. An implant reducer as recited in claim 1, wherein the first arm includes a first pivot configured to facilitate rotation of the second member of the first arm relative to the first member of the first arm and the second arm includes a second pivot configured to facilitate rotation of the second member of the second arm relative to the first member of the second arm.

6. An implant reducer as recited in claim 1, wherein the connector includes a biasing member.

7. An implant reducer as recited in claim 1, wherein the connector includes a biasing member configured to resiliently bias the connector from the third configuration to the second configuration.

8. An implant reducer as recited in claim 1, wherein the connector includes a spring.

9. An implant reducer as recited in claim 1, wherein the first implant engaging portion includes at least one projection configured to engage an implant.

10. An implant reducer as recited in claim 1, wherein the second implant engaging portion includes at least one concave surface configured to engage an implant.

11. An implant reducer as recited in claim 1, further comprising a handle including the first members, the handle including a lock configured to fix the arms in a selected configuration.

12. A spinal implant system comprising:
an implant reducer comprising:
a first arm including a proximal member and a distal member, the distal member including an inner surface that defines a longitudinal cavity and a first implant engaging portion, and
a second arm including a proximal member and a distal member, the distal member of the second arm including a connector and a second implant engaging portion, the proximal members being connected and the connector being movably disposed in the longitudinal cavity;
a first implant including a proximal portion defining an implant cavity and a distal portion configured for penetrating tissue, the first implant engaging portion being configured to engage the proximal portion of the first implant; and
a second implant including a vertebral rod configured for disposal with the implant cavity, the second implant engaging portion being configured to engage the second implant,
wherein the first members are relatively rotatable to move the arms between at least
a first configuration such that the first implant engaging portion and the second implant engaging portion are spaced apart,
a second configuration such that the first implant engaging portion engages the proximal portion of the first implant and is disposed adjacent the second implant engaging portion and
a third configuration such that the connector translates relative to the inner surface and the second implant engaging portion translates relative to the first implant engaging portion.

13. An implant reducer as recited in claim 12, wherein the distal members are connected via a pivot configured to facilitate relative rotation of the proximal members.

14. An implant reducer as recited in claim 12, wherein the first arm includes a first pivot configured to facilitate rotation of the distal member of the first arm relative to the proximal member of the first arm and the second arm includes a second pivot configured to facilitate rotation of the distal member of the second arm relative to the proximal member of the second arm.

15. An implant reducer as recited in claim 12, wherein the connector includes a biasing member.

16. An implant reducer as recited in claim 12, wherein the connector includes a biasing member configured to resiliently bias the connector from the third configuration to the second configuration.

17. The spinal rod system of claim 12, wherein the proximal portion of the first implant includes an outer surface that defines an axial cavity disposed adjacent a proximal end of the proximal portion, the axial cavity being configured for receiving the first implant engaging portion.

18. The spinal rod system of claim 12, wherein the proximal portion of the first implant includes an outer surface that defines an axial cavity including a transverse opening configured for passage of the first implant engaging portion.

19. The spinal rod system of claim 12, wherein the translation of the second implant engaging portion relative to the first implant engaging portion causes rotation of the implant cavity relative to the vertebral rod.

20. A spinal implant system comprising:
a rod reducer comprising:
a first arm including a proximal member connected to a distal member via a first pivot such that the distal member is rotatable relative to the proximal member, the distal member including an inner surface that defines a longitudinal slot and a pair of spaced apart extensions including inwardly disposed pins, and
a second arm including a proximal member connected to a distal member via a second pivot such that the distal member of the second arm is rotatable relative to the proximal member of the second arm, the distal member of the second arm including a connector and a pair of spaced apart extensions including concave surfaces, the proximal members being connected and the connector being movably disposed in the longitudinal slot;
a bone fastener including a receiver defining an implant cavity and a threaded distal shaft, the receiver further including an outer surface that defines an axial cavity including a transverse opening configured for passage of the pins; and
a vertebral rod configured for disposal with the implant cavity, the concave surfaces configured to engage an outer surface of the vertebral rod,
wherein the proximal members are relatively rotatable to move the arms between
a first configuration such that the extensions of the arms are spaced apart,
a second configuration such that the pins are disposed in the axial cavity and the concave surfaces engage the outer surface of the vertebral rod, and the extensions of the first arm are disposed adjacent the extensions of the second arm, and
a third configuration such that the connector translates relative to the inner surface and the extensions of the first arm translate relative to the extensions of the second arm such that the implant cavity rotates relative to the vertebral rod.
